SEQUENCE LISTING (1 sequence)

SEQ ID NO: 1 - protein, 303 aa, Unknown
  Description of Unknown exemplary polypeptide sequence
SKNFHLRPRDLISNINVIVLELKGSETTFMCEYADETATIVEFLNRWITFSQSIISTLTGGSSSTKKTQLQLEHLLLDLQMILNGINNYKNPKLTRMLTFKFYMPKKATELKHLQCLEEELKPLEEVLNLAQGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

(19) United States
(12) Patent Application Publication
(10) Pub. No.: US 2021/0395325 A1
(43) Pub. Date: Dec. 23, 2021

(54) IL-2 FUSION POLYPEPTIDE COMPOSITIONS AND METHODS OF MAKING AND USING THE SAME

(71) Applicant: Alkermes Pharma Ireland Limited, Dublin (IE)

(72) Inventors: Francisca O. Gbormittah, Acton, MA (US); Tarek A. Zeidan, Lexington, MA (US)

(21) Appl. No.: 17/315,974

(22) Filed: May 10, 2021

Related U.S. Application Data
(60) Provisional application No. 63/022,860, filed on May 11, 2020.

(51) Int. Cl. C07K 14/55; A61K 47/26; A61K 47/10

(52) U.S. Cl. CPC C07K 14/55; A61K 38/00; A61K 47/10; A61K 47/26

(57) ABSTRACT

Provided herein are compositions comprising polypeptides comprising a circularly permuted interleukin-2 (IL-2) fused to the extracellular portion of an IL-2Rα chain, and methods of making and using such compositions.

### CROSS-REFERENCE TO RELATED APPLICATION

This application claims the benefit of U.S. Provisional Application Ser. No. 63/022,860, filed May 11, 2020, the entire disclosure of which is incorporated herein by reference.

### FIELD OF THE INVENTION

This disclosure relates to compositions comprising polypeptides comprising a circularly permuted interleukin-2 (IL-2) fused to the extracellular portion of an IL-2Rα chain, and methods of making and using such compositions.

### BACKGROUND

Polypeptides comprising a circularly permuted interleukin-2 (IL-2) fused to the extracellular portion of an IL-2Rα chain interleukin-2 (IL-2) interleukin-2 receptor alpha (IL-2Rα) hold great promise as anti-cancer agents. These polypeptide s retain full ability to signal through the intermediate-affinity IL-2R complex that is expressed on memory CD8+ T cells and Natural Killer (NK) cells, but are sterically prevented from binding to the high-affinity IL-2R complex that is preferentially expressed on CD4+ FOXP3+ regulatory T cells (CD4+ Tregs) and endothelial cells. As a result of this selective IL-2R binding, the polypeptides selectively activate CD8+ T cells and NK cells, thereby promoting tumor cell killing. The inability to activate the high-affinity IL-2R on endothelial cells may also reduce the risk of toxicity due to capillary leak syndrome, a known risk of IL-2 therapies.

When used for the treatment of human subjects, the aforementioned polypeptides must be stored prior to use and transported to the point of administration. Reproducibly attaining a desired level of polypeptide in a subject requires that the polypeptide be stored in a formulation that maintains the bioactivity of the polypeptide. Accordingly, there is a need in the art for stable compositions of polypeptides. Preferably, such compositions will exhibit a long shelf-life, and be stable when stored and transported.

### SUMMARY

The present disclosure provides compositions comprising polypeptides comprising a circularly permuted IL-2 fused to the extracellular portion of an IL-2Rα chain, and methods of making and using such compositions. These compositions are specifically formulated to improve the stability and shelf-life of the polypeptides contained therein.

In one aspect, the disclosure provides a composition comprising:
a) about 1 mg to about 50 mg of a polypeptide comprising a circularly permuted IL-2 fused to the extracellular portion of an IL-2Rα chain;
b) sucrose;
c) mannitol;
c) citrate buffer; and
d) an emulsifier.

In certain embodiments, the polypeptide comprises an amino acid sequence having at least 95% identity to SEQ ID NO: 1. In certain embodiments, the polypeptide comprises the amino acid sequence of SEQ ID NO: 1. In certain embodiments, the polypeptide consists of the amino acid sequence of SEQ ID NO: 1.

In certain embodiments, the composition comprises about 1 mg to about 15 mg of the polypeptide. In certain embodiments, the composition comprises about 1 mg of the polypeptide. In certain embodiments, the composition comprises about 5 mg of the polypeptide. In certain embodiments, the composition comprises about 15 mg of the polypeptide. In certain embodiments, the composition comprises about 20 mg of the polypeptide. In certain embodiments, the composition comprises about 30 mg of the polypeptide.

In certain embodiments, the composition comprises about 60 mg to about 72 mg sucrose. In certain embodiments, the composition comprises about 66 mg sucrose.

In certain embodiments, the composition comprises about 60 mg to about 72 mg mannitol. In certain embodiments, the composition comprises about 66 mg mannitol.

In certain embodiments, the composition comprises about 4.0 mg to about 6.0 mg citrate anion. In certain embodiments, the composition comprises about 5.0 mg citrate anion.

In certain embodiments, the composition comprises citric acid and sodium citrate tribasic dihydrate in a mass ratio of citric acid:sodium citrate tribasic dihydrate of between about 1:10 to about 1:2 (i.e., about 1:10, about 1:9, about 1:8, about 1:7, about 1:6, about 1:5, about 1:4, about 1:3, and about 1:2).

In certain embodiments, the composition comprises citric acid and sodium citrate tribasic dihydrate in a mass ratio of citric acid:sodium citrate tribasic dihydrate of about 1:9. In certain embodiments, the composition comprises citric acid and sodium citrate tribasic dihydrate in a mass ratio of citric acid:sodium citrate tribasic dihydrate of about 1:2.

In certain embodiments, the emulsifier comprises polysorbate 20. In certain embodiments, the composition comprises about 0.20 mg to about 0.24 mg polysorbate 20. In certain embodiments, the composition comprises about 0.22 mg polysorbate 20.

In certain embodiments, the composition comprises about 0.10 mg to about 0.12 mg polysorbate 20. In certain embodiments, the composition comprises about 0.11 mg polysorbate 20.

In certain embodiments, the composition is a lyophilized cake.

In certain embodiments, dissolution of the lyophilized cake in water results in an aqueous solution with a pH of about 5.5 to about 6.5. In certain embodiments, dissolution of the lyophilized cake in water results in an aqueous solution with a pH of about 6.1.

In certain embodiments, dissolution of the lyophilized cake in water results in an aqueous solution with an isotonic osmolality. In certain embodiments, dissolution of the lyophilized cake in water results in an aqueous solution with an osmolality of about 240 to about 340 mOsm/kg. In certain embodiments, dissolution of the lyophilized cake in water results in an aqueous solution with an osmolality of about 280 to about 320 mOsm/kg. In certain embodiments, dissolution of the lyophilized cake in water results in an aqueous solution with an osmolality of about 285 mOsm/kg. In certain embodiments, dissolution of the lyophilized cake in water results in an aqueous solution with an osmolality of about 300 mOsm/kg.

In certain embodiments, the composition is an aqueous solution.

In certain embodiments, the aqueous solution comprises about 0.03 mg/mL of the polypeptide to about 0.2 mg/mL of the polypeptide.

In certain embodiments, the composition comprises about 0.5 mg/mL to about 30 mg/mL of the polypeptide.

In certain embodiments, the composition comprises about 1 mg/mL of the polypeptide.

In certain embodiments, the composition comprises about 5 mg/mL of the polypeptide.

In certain embodiments, the composition comprises about 15 mg/mL of the polypeptide.

In certain embodiments, the composition comprises about 20 mg/mL of the polypeptide.

In certain embodiments, the composition comprises about 30 mg/mL of the polypeptide.

In certain embodiments, the composition comprises about 25 mg/mL to about 35 mg/mL sucrose. In certain embodiments, the composition comprises about 30 mg/mL sucrose.

In certain embodiments, the composition comprises about 25 mg/mL to about 35 mg/mL mannitol. In certain embodiments, the composition comprises about 30 mg/mL mannitol.

In certain embodiments, the composition comprises about 10 mM to about 20 mM citrate buffer. In certain embodiments, the composition comprises about 12 mM citrate buffer. In certain embodiments, the citrate buffer is formed by the combination of 2.03 mg/mL sodium citrate tribasic dihydrate and 0.97 mg/mL citric acid monohydrate in the aqueous solution. In certain embodiments, the citrate buffer is formed by the combination of 2.91 mg/mL sodium citrate tribasic dihydrate and 0.34 mg/mL citric acid monohydrate in the aqueous solution. In certain embodiments, the citrate buffer is formed by the combination of 2.96 mg/mL sodium citrate tribasic dihydrate and 0.30 mg/mL citric acid monohydrate in the aqueous solution.

In certain embodiments, the composition comprises about 0.09 mg/mL to about 0.11 mg/mL polysorbate 20. In certain embodiments, the composition comprises about 0.1 mg/mL polysorbate 20.

In certain embodiments, the pH of the composition is about 5.5 to about 6.5. In certain embodiments, the pH of the composition is about 6.1.

In certain embodiments, the osmolality of the composition is about 240 to about 340 mOsm/kg. In certain embodiments, the osmolality of the composition is about 280 to about 320 mOsm/kg. In certain embodiments, the osmolality of the composition is about 285 mOsm/kg. In certain embodiments, the osmolality of the composition is about 300 mOsm/kg.

In certain embodiments, the composition is a single unit dose of the polypeptide.

In one aspect, the disclosure provides an aqueous composition comprising:
a) about 1 mg/mL to about 30 mg/mL of a polypeptide comprising a circularly permuted IL-2 fused to the extracellular portion of an IL-2Rα chain;
b) about 25 mg/mL to about 35 mg/mL sucrose;
c) about 25 mg/mL to about 35 mg/mL mannitol;
d) about 10 mM to about 20 mM citrate buffer; and
e) about 0.09 mg/mL to about 0.11 mg/mL polysorbate 20,
wherein the pH of the solution is about 5.5 to about 6.5.

In certain embodiments, the polypeptide comprises an amino acid sequence having at least 95% identity to SEQ ID NO: 1. In certain embodiments, the polypeptide comprises the amino acid sequence of SEQ ID NO: 1.

In one aspect, the disclosure provides an aqueous composition comprising:
a) about 1 mg/mL to about 30 mg/mL of a polypeptide comprising the amino acid sequence of SEQ ID NO: 1;
b) about 25 mg/mL to about 35 mg/mL sucrose;
c) about 25 mg/mL to about 35 mg/mL mannitol;
d) about 10 mM to about 20 mM citrate buffer; and
e) about 0.09 mg/mL to about 0.11 mg/mL polysorbate 20,
wherein the pH of the solution is about 5.5 to about 6.5.

In certain embodiments, the composition comprises about 30 mg/mL sucrose.

In certain embodiments, the composition comprises about 30 mg/mL mannitol.

In certain embodiments, the composition comprises about 12 mM citrate buffer.

In certain embodiments, the composition comprises about 0.11 mg/mL polysorbate 20.

In certain embodiments, the pH of the solution is about 6.1.

In certain embodiments, the composition comprises about 1 mg/mL of the polypeptide. In certain embodiments, the composition comprises about 5 mg/mL of the polypeptide. In certain embodiments, the composition comprises about 15 mg/mL of the polypeptide. In certain embodiments, the composition comprises about 20 mg/mL of the polypeptide. In certain embodiments, the composition comprises about 30 mg/mL of the polypeptide.

In another aspect the disclosure provides an aqueous composition comprising:
a) about 1, 5, 15, or 30 mg/mL of a polypeptide comprising the amino acid sequence of SEQ ID NO: 1;
b) about 25 mg/mL to about 35 mg/mL sucrose;
c) about 25 mg/mL to about 35 mg/mL sucrose;
d) about 8 mM citrate buffer to about 14 mM citrate buffer (e.g., 8 mM, 9 mM, 10 mM, 11 mM, 12 mM, 13 mM, or 14 mM); and
e) about 0.1 mg/mL polysorbate 20,
wherein the pH of the composition is about 6.1.

In another aspect the disclosure provides an aqueous composition comprising:
a) about 1, 5, 15, or 30 mg/mL of a polypeptide comprising the amino acid sequence of SEQ ID NO: 1;
b) about 25 mg/mL to about 35 mg/mL sucrose;
c) about 25 mg/mL to about 35 mg/mL sucrose;
d) about 12 mM citrate buffer; and
e) about 0.1 mg/mL polysorbate 20,
wherein the pH of the composition is about 6.1.

In another aspect the disclosure provides an aqueous composition comprising:
a) about 1, 5, 15, or 30 mg/mL of a polypeptide comprising the amino acid sequence of SEQ ID NO: 1;
b) about 25 mg/mL to about 35 mg/mL sucrose;
c) about 25 mg/mL to about 35 mg/mL sucrose;
d) about 2 mg/mL sodium citrate tribasic dihydrate;
e) about 1 mg/mL citric acid monohydrate; and
f) about 0.1 mg/mL polysorbate 20,
wherein the pH of the composition is about 6.1.

In another aspect the disclosure provides an aqueous composition comprising:
a) about 1, 5, 15, or 30 mg/mL of a polypeptide comprising the amino acid sequence of SEQ ID NO: 1;
b) about 25 mg/mL to about 35 mg/mL sucrose;
c) about 25 mg/mL to about 35 mg/mL sucrose;
d) about 2.03 mg/mL sodium citrate tribasic dihydrate;
e) about 0.97 mg/mL citric acid monohydrate; and
f) about 0.1 mg/mL polysorbate 20,
wherein the pH of the composition is about 6.1.

In another aspect the disclosure provides an aqueous composition comprising:
a) about 1, 5, 15, or 30 mg/mL of a polypeptide comprising the amino acid sequence of SEQ ID NO: 1;
b) about 25 mg/mL to about 35 mg/mL sucrose;
c) about 25 mg/mL to about 35 mg/mL sucrose;
d) about 3 mg/mL sodium citrate tribasic dihydrate;
e) about 0.3 mg/mL citric acid monohydrate; and
f) about 0.1 mg/mL polysorbate 20,
wherein the pH of the composition is about 6.1.

In another aspect the disclosure provides an aqueous composition comprising:
a) about 1, 5, 15, or 30 mg/mL of a polypeptide comprising the amino acid
sequence of SEQ ID NO: 1;
b) about 25 mg/mL to about 35 mg/mL sucrose;
c) about 25 mg/mL to about 35 mg/mL sucrose;
d) about 2.91 mg/mL sodium citrate tribasic dihydrate;
e) about 0.34 mg/mL citric acid monohydrate; and
f) about 0.1 mg/mL polysorbate 20,
wherein the pH of the composition is about 6.1.

In another aspect, the disclosure provides an article of manufacture comprising any of the foregoing compositions. In certain embodiments, the article is a glass vial.

In another aspect, the disclosure provides a lyophilized composition made by lyophilizing any of the foregoing aqueous solutions.

In another aspect, the disclosure provides a method of making a lyophilized composition, the method comprising lyophilizing any of the foregoing aqueous solutions.

In another aspect, the disclosure provides a method of making an aqueous composition, the method comprising dissolving any of the foregoing lyophilized compositions in an aqueous solvent. In certain embodiments, the aqueous solvent is water for injection. In certain embodiments, the aqueous solvent is a sodium chloride solution.

In certain embodiments, the pH of the aqueous composition is adjusted to about 6.1. In certain embodiments, the pH of the aqueous composition is adjusted to about 6.1 with a base. In certain embodiments, the base is sodium hydroxide.

In certain embodiments, the aqueous composition is further diluted with an aqueous solution comprising about 1% (w/w) of a surfactant. In certain embodiments, the surfactant is polysorbate 20. In certain embodiments, the aqueous solution further comprises about 0.1% (w/w) citric acid monohydrate, 0.2% (w/w) sodium citrate tribasic dihydrate, and 98.7% (w/w) water for injection.

In certain embodiments, the composition comprises a pharmaceutical composition.

In another aspect, the disclosure provides a method of activating natural killer cells (NK) cells in a subject, the method comprising administering to the subject an effective amount of any of the foregoing compositions.

In another aspect, the disclosure provides a method of treating cancer in a subject in need thereof, the method comprising administering to the subject an effective amount of any of the foregoing compositions. In certain embodiments, the cancer is renal cell carcinoma, melanoma, ovarian cancer, or lung cancer. In certain embodiments, the cancer comprises a refractory solid tumor.

### DETAILED DESCRIPTION

Provided herein are compositions comprising polypeptides comprising a circularly permuted IL-2 fused to the extracellular portion of an IL-2Rα chain, and methods of making and using such compositions.

The formulations disclosed herein provide improved stability and shelf-life of the polypeptides contained therein. In particular, the polypeptide product retains biological activity, including after being lyophilized in the recited formulations and reconstituted in water for injection (WFI) or a similarly acceptable diluent. Importantly, the formulations described herein have been designed to allow the lyophilized product to be reconstituted in WFI, with is readily available to a patient or healthcare provider. When reconstituted in WFI, the formulations described herein possess a physiologically-acceptable osmolality, allowing the reconstituted product to be administered subcutaneously. This eliminates the need for a specialized diluent to reconstitute the lyophilized product with an appropriate osmolality, making it easier for a patient or healthcare provider to use the drug and, therefore, improve drug use compliance.

Subcutaneous administration has advantages for drug delivery as well. When delivered via the subcutaneous route, the drug may be delivered more quickly compared to other delivery routes (e.g., intravenous). Subcutaneous delivery may also be performed by a patient in their home, rather than by a healthcare provider in a healthcare facility. This patient-directed delivery may also improve drug use compliance.

The formulations provided herein also yield a lyophilized cake that has a preferred appearance. Specifically, the cake is intact (not fragmented), has little to no shrinkage from the container (e.g., a glass vial), and have an even, concave surface.

### Selected Definitions

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the terms “comprising,” “including,” “having,” and grammatical variants thereof are to be taken as specifying the stated features, integers, steps or components but do not preclude the addition of one or more additional features, integers, steps, components or groups thereof. These terms encompass the terms “consisting of” and “consisting essentially of.”

As used herein, the terms “circular permutation” and “circularly permuted” refer to the process of taking a linear protein, or its cognate nucleic acid sequence, and fusing the native N- and C-termini (directly or through a linker, using protein or recombinant DNA methodologies) to form a circular molecule, and then cutting (opening) the circular molecule at a different location to form a new linear protein, or cognate nucleic acid molecule, with termini different from the termini in the original molecule. Circular permutation thus preserves the sequence, structure, and function of a protein, while generating new C- and N-termini at different locations that results in an improved orientation for fusing a desired polypeptide fusion partner as compared to the original molecule.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of up to ±5%, including ±5%, ±1%, and ±0.1% from the specified value, as such variations are appropriate to perform the disclosed methods.

As used herein, the terms “treat,” “treated,” “treating,” or “treatment” include the diminishment or alleviation of at least one symptom associated or caused by the state, disorder or disease being treated.

As used herein, the term “effective amount” in the context of the administration of a therapy to a subject refers to the amount of a therapy that achieves a desired prophylactic or therapeutic effect.

As used herein, the term “patient,” “individual” or “subject” refers to a human or a non-human mammal. Non-human mammals include, for example, livestock and pets, such as ovine, bovine, porcine, canine, feline and murine mammals. In certain embodiments, the subject is a human.

### IL-2 Fusion Polypeptides

In one aspect, the instant disclosure provides compositions of polypeptides comprising a circularly permuted interleukin-2 (IL-2) fused to the extracellular portion of an IL-2Rα chain. The polypeptides employed in the compositions disclosed herein exhibit preferential binding to the intermediate-affinity IL-2R complex comprising IL-2R0 and the common gamma chain, IL-2Ry) relative to the high-affinity IL-2R complex (comprising IL-2Rα, IL-2R13, and IL-2Ry), and behave as selective agonists of the intermediate-affinity IL-2R complex. The design and generation of such polypeptides is described in U.S. Pat. No. 9,359,415, which is hereby incorporated by reference in its entirety.

An exemplary polypeptide useful for inclusion in the compositions disclosed herein is set forth below in SEQ ID. NO:1:

(SEQ ID NO: 1)
SKNFHLRPRDLISNINVIVLELKGSETTFMCEYADETATIV
EFLNRWITFSQSIISTLTGGSSSTKKTQLQLEHLLLDLQM
ILNGINNYKNPKLTRMLTFKFYMPKKATELKHLQCLEEEL
KPLEEVLNLAQGSGGGSELCDDDPPEIPHATFKAMAYKEG
TMLNCECKRGFRRIKSGSLYMLCTGNSSHSSWDNQCQCTS
SATRNTTKQVTPQPEEQKERKTTEMQSPMQPVDQASLPGH
CREPPPWENEATERIYHFVVGQMVYYQCVQGYRALHRGPA
ESVCKMTHGKTRWTQPQLICTG

Accordingly, in certain embodiments, the amino acid sequence of the polypeptide comprises the amino acid sequence of SEQ ID. NO: 1. In certain embodiments, the amino acid sequence of the polypeptide consists of the amino acid sequence of SEQ ID. NO: 1.

The skilled worker will appreciate that amino acid sequence variants of SEQ ID. NO: 1 can also be employed in the compositions disclosed herein. For example, in certain embodiments, the amino acid sequence of the polypeptide comprises or consists of an amino acid sequence having at least 80% (e.g., 80, 81, 82, 83, 84, 85, 86, 87, 88, 89, 90, 91, 92, 93, 94, 95, 96, 97, 98, or 99%) identity to the amino acid sequence of SEQ ID. NO:1. In certain embodiments, the amino acid sequence of the polypeptide comprises or consists of an amino acid sequence having at least 95% identity to the amino acid sequence of SEQ ID. NO:1.

The skilled worker will also appreciate that amino acid sequence of the polypeptides employed in the compositions disclosed herein can be derivatized or modified, e.g., pegylated, amidated, etc.

In certain embodiments, the amount of the polypeptide in a formulation is about 1 mg to about 50 mg (e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 40 mg, about 44 mg, about 45 mg, or about 50 mg). In certain embodiments, the amount of the polypeptide is about 1 mg to about 30 mg. In certain embodiments, the amount of the polypeptide is about 1 mg to about 15 mg. In certain embodiments, the amount of the polypeptide is about 1 mg. In certain embodiments, the amount of the polypeptide is about 2.2 mg. In certain embodiments, the amount of the polypeptide is about 5 mg. In certain embodiments, the amount of the polypeptide is about 11 mg. In certain embodiments, the amount of the polypeptide is about 15 mg. In certain embodiments, the amount of the polypeptide is about 20 mg. In certain embodiments, the amount of the polypeptide is about 30 mg. In certain embodiments, the amount of the polypeptide is about 44 mg.

In certain embodiments, the concentration of the polypeptide in an aqueous formulation is about 0.5 mg/mL to about 50 mg/mL. In certain embodiments, the concentration of the polypeptide is about 0.5 mg/mL to about 20 mg/mL (e.g., about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 25 mg/mL, about 30 mg/mL, about 35 mg/mL, about 40 mg/mL, about 45 mg/mL, or about 50 mg/mL). In certain embodiments, the concentration of the polypeptide is about 1 mg/mL. In certain embodiments, the concentration of the polypeptide is about 5 mg/mL. In certain embodiments, the concentration of the polypeptide is about 15 mg/mL. In certain embodiments, the concentration of the polypeptide is about 20 mg/mL. In certain embodiments, the concentration of the polypeptide is about 30 mg/mL.

### Excipients & Buffers

In certain embodiments, the compositions disclosed herein comprise one or more excipients and/or buffers.

As used herein, the term “excipient” refers to any non-therapeutic agent added to the composition or formulation to provide a desired consistency, viscosity, or stabilizing effect. Suitable excipients for use in the compositions disclosed herein can act, e.g., as viscosity enhancing agents, stabilizers, solubilizing agents, etc. The excipient can be ionic or non-ionic. Suitable ionic excipients include salts such as NaCl or amino acid components such as arginine-HCl. Suitable non-ionic excipients include sugars, for example, monosaccharides (e.g., fructose, maltose, galactose, glucose, D-mannose, sorbose, etc.); disaccharides (e.g., lactose, sucrose, trehalose, cellobiose, etc.); polysaccharides (e.g., raffinose, melezitose, maltodextrins, dextrans, starches, etc.); and sugar alcohols (e.g., mannitol, xylitol, maltitol, lactitol, xylitol sorbitol (glucitol), etc.). For example, the sugar may be sucrose, trehalose, raffinose, maltose, sorbitol or mannitol. Additionally or alternatively, the sugar may be a sugar alcohol or an amino sugar. In certain embodiments, the sugar is sucrose and mannitol.

In certain embodiments, the amount of the excipient (e.g., sucrose and mannitol) in a formulation is about 1 mg to about 150 mg (e.g., about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg). In certain embodiments, the amount of the excipient (e.g., sucrose and mannitol) in a formulation is about 30 mg to about 90 mg. In certain embodiments, the amount of the excipient (e.g., sucrose and mannitol) in a formulation is about 60 mg to about 72 mg. In certain embodiments, the amount of the excipient (e.g., sucrose and mannitol) in a formulation is about 66 mg.

In certain embodiments, the concentration of the excipient (e.g., sucrose and mannitol) in an aqueous formulation is about 1 mg/mL to about 100 mg/mL (e.g., about 1 mg/mL, about 10 mg/mL, about 20 mg/mL, about 30 mg/mL, about 40 mg/mL, about 45 mg/mL, about 50 mg/mL, about 55 mg/mL, about 60 mg/mL, about 70 mg/mL, about 80 mg/mL, about 90 mg/mL, or about 100 mg/mL). In certain embodiments, the concentration of excipient (e.g., sucrose and mannitol) is about 10 mg/mL to about 50 mg/mL. In certain embodiments, the concentration of excipient (e.g., sucrose and mannitol) is about 25 mg/mL to about 35 mg/mL. In certain embodiments, the concentration of the excipient (e.g., sucrose and mannitol) is about 30 mg/mL.

Suitable buffering agents for use in the compositions disclosed herein include organic acid and salts, such as salts of citric acid, ascorbic acid, gluconic acid, carbonic acid, tartaric acid, succinic acid, acetic acid or phthalic acid; Tris, tromethamine hydrochloride, or phosphate buffer. In addition, amino acid components can also be used as buffering agents. Such amino acid component includes glycine, histidine, and methionine. In certain embodiments, the buffer is a citrate buffer. As used herein, the term “citrate buffer” refers to a pH buffering system (in aqueous or lyophilized form) that utilizes citrate ions. Citrate buffer can be made using any art recognized methods, including, by combining: (i) citric acid, trisodium citrate dihydrate, and citric acid monohydrate; or (ii) citric acid monohydrate, sodium phosphate dibasic, and citric acid. In certain embodiments, citrate buffer is made using sodium citrate dihydrate and citric acid.

In certain embodiments, the amount of the buffering agent (e.g., citrate) in the formulation is about 1 mg to about 10 mg (e.g., about 1 mg, about 2 mg, about 3 mg, about 4 mg, about 5 mg, about 6 mg, about 7 mg, about 8 mg, about 9 mg, about 10 mg). In certain embodiments, the amount of the buffering agent (e.g., sodium citrate) is about 5.9 mg to about 7.2 mg (e.g., about 5.9 mg, about 6.0 mg, about 6.1 mg, about 6.2 mg, about 6.3 mg, about 6.4 mg, about 6.5 mg, about 6.6 mg about, 6.7 mg about 6.8 mg, about 6.9 mg, about 7.0 mg, about 7.1 mg, or about 7.2 mg). In certain embodiments, the amount of the buffering agent (e.g., citrate) is about 6.6 mg. In certain embodiments, the amount of the citrate anion in the buffering agent (e.g., citrate) is about 4.0 mg to about 6.0 mg. In certain embodiments, the amount of the citrate anion in the buffering agent (e.g., citrate) is about 5.0 mg.

In certain embodiments, the concentration of the buffering agent (e.g., citrate) in an aqueous formulation disclosed herein is about 1 mM to about 50 mM (e.g., about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, or about 50 mM). In certain embodiments, the concentration of the buffering agent (e.g., sodium citrate) is about 11 mM to about 13 mM (e.g., about 11.1 mM, 11.2 mM, 11.3 mM, 11.4 mM, 11.5 mM, 11.6 mM, 11.7 mM, 11.8 mM, 11.9 mM, 12.1 mM, 12.2 mM, 12.3 mM, 12.4 mM, 12.5 mM, 12.6 mM, 12.7 mM, 12.8 mM, or 12.9 mM). In certain embodiments, the concentration of the buffering agent (e.g., citrate) is about 12 mM. In certain embodiments, the concentration of the buffering agent (e.g., citrate) is about 11.95 mM. In certain embodiments, the concentration of the buffering agent (e.g., citrate) is about 11.67 mM. In certain embodiments, the citrate buffer contains 2.03 mg/mL (6.90 mM) sodium citrate tribasic dihydrate and 0.97 mg/mL (5.05 mM) citric acid. In certain embodiments, the citrate buffer contains 2.91 mg/mL (9.90 mM) sodium citrate tribasic dihydrate and 0.34 mg/mL (1.77 mM) citric acid.

In certain embodiments, the compositions disclosed herein have a pH of about 5.0 to about 8.0, of about 5.5 to about 7.5, of about 5.0 to about 7.0, of about 6.0 to about 8.0, or of about 6.0 to about 7.0. In certain embodiments, the compositions have a pH of about 5.4 to about 6.5. In certain embodiments, the compositions have a pH of about 5.8 to about 6.4. In certain embodiments, the compositions have a pH of about 6.1. In certain embodiments, the pH of the composition is adjusted to a pH of about 6.1. In certain embodiment, the pH is adjusted with a base. In certain embodiments, the base is a hydroxide salt, such as sodium hydroxide (NaOH) or potassium hydroxide (KOH). In certain embodiments, the composition is an aqueous composition and the pH of the aqueous composition is adjusted to a pH of about 6.1.

In certain embodiments, the compositions disclosed herein have an isotonic osmolality. In certain embodiments, osmolality of the composition is about 240 to about 340 mOsm/kg. In certain embodiments, osmolality of the composition is about 280 to about 320 mOsm/kg. In certain embodiments, the osmolality of the composition is about 285 mOsm/kg. In certain embodiments, the osmolality of the composition is about 300 mOsm/kg.

As used herein, the term “surfactant” refers to organic substances having amphipathic structures; i.e., they are composed of groups of opposing solubility tendencies, typically an oil-soluble hydrocarbon chain and a water-soluble ionic group. Surfactants can be classified, depending on the charge of the surface-active moiety, into anionic, cationic and dispersing agents for various pharmaceutical compositions and preparations of biological materials. Suitable surfactants for use in the compositions disclosed herein include non-ionic surfactants, ionic surfactants and zwitterionic surfactants. Typical surfactants for use with the invention include sorbitan fatty acid esters (e.g., sorbitan monocaprylate, sorbitan monolaurate, sorbitan monopalmitate), sorbitan trioleate, glycerine fatty acid esters (e.g., glycerine monocaprylate, glycerine monomyristate, glycerine monostearate), polyglycerine fatty acid esters (e.g., decaglyceryl monostearate, decaglyceryl distearate, decaglyceryl monolinoleate), polyoxyethylene sorbitan fatty acid esters (e.g., polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g., polyoxyethylene sorbitol tetrastearate, polyoxyethylene sorbitol tetraoleate), polyoxyethylene glycerine fatty acid esters (e.g., polyoxyethylene glyceryl monostearate), polyethylene glycol fatty acid esters (e.g., polyethylene glycol distearate), polyoxyethylene alkyl ethers (e.g., polyoxyethylene lauryl ether), polyoxyethylene polyoxypropylene alkyl ethers (e.g., polyoxyethylene polyoxypropylene glycol, polyoxyethylene polyoxypropylene propyl ether, polyoxyethylene polyoxypropylene cetyl ether), polyoxyethylene alkylphenyl ethers (e.g., polyoxyethylene nonylphenyl ether), polyoxyethylene hydrogenated castor oils {e.g., polyoxyethylene castor oil, polyoxyethylene hydrogenated castor oil), polyoxyethylene beeswax derivatives (e.g., polyoxyethylene sorbitol beeswax), polyoxyethylene lanolin derivatives (e.g., polyoxyethylene lanolin), and polyoxyethylene fatty acid amides (e.g., polyoxyethylene stearic acid amide); C 10-C 18 alkyl sulfates (e.g., sodium cetyl sulfate, sodium lauryl sulfate, sodium oleyl sulfate), polyoxyethylene C 10-C 18 alkyl ether sulfate with an average of 2 to 4 moles of ethylene oxide units added (e.g., sodium polyoxyethylene lauryl sulfate), and C1-C 18 alkyl sulfosuccinate ester salts (e.g., sodium lauryl sulfosuccinate ester); and natural surfactants such as lecithin, glycerophospholipid, sphingophospholipids (e.g., sphingomyelin), and sucrose esters of C 12-C 18 fatty acids. A composition may include one or more of these surfactants. In certain embodiments, the compositions disclosed herein comprise polyoxyethylene sorbitan fatty acid esters e.g., polysorbate 20, 40, 60 or 80. In certain embodiments, the compositions disclosed herein comprise polysorbate 20.

In certain embodiments, the amount of the surfactant (e.g., polysorbate 20) in the formulation is about 0.1 mg to about 1 mg (e.g., about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, or about 1 mg). In certain embodiments, the amount of the surfactant (e.g., polysorbate 20) is about 0.15 mg to about 0.3 mg (e.g., about 0.16 mg, about 0.17 mg, about 0.18 mg, about 0.19 mg, about 0.21 mg, about 0.22 mg, about 0.23 mg, about 0.24 mg, about 0.26 mg, about 0.27 mg, about 0.28 mg, or about 0.29 mg). In certain embodiments, the amount of the surfactant (e.g., polysorbate 20) is about 0.20 mg to about 0.24 mg. In certain embodiments, the amount of the surfactant (e.g., polysorbate 20) in an aqueous formulation is about 0.22 mg. In certain embodiments, the composition comprises about 0.10 mg to about 0.12 mg polysorbate 20. In certain embodiments, the composition comprises about 0.11 mg polysorbate 20.

In certain embodiments, the concentration of the surfactant (e.g., polysorbate 20) in an aqueous formulation is about 0.01 mg/mL to about 1 mg/mL (e.g., about 0.01 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1 mg/mL). In certain embodiments, the concentration of the surfactant (e.g., polysorbate 20) is about 0.05 mg/mL to about 0.15 mg/mL (e.g., about 0.05 mg/mL, about 0.06 mg/mL, about 0.07 mg/mL, or about 0.08 mg/mL about 0.09 mg/mL, about 0.1 mg/mL, about 0.11 mg/mL, about 0.12 mg/mL, about 0.13 mg/mL, about 0.14 mg/mL, or about 0.15 mg/mL). In certain embodiments, the concentration of the surfactant (e.g., polysorbate 20) is about 0.09 mg/mL to about 0.11 mg/mL. In certain embodiments, the concentration of the surfactant (e.g., polysorbate 20) in an aqueous formulation is about 0.1 mg/mL.

It will be understood to those of skill in the art that the components of the compositions and compositions of the present invention may be described by units other than mg/mL. For example, the components of the compositions and compositions of the present invention may be described in units of molarity. The components of the compositions and compositions of the present invention may be further described in units of weight or mass percent.

### Lyophilization

In one aspect, the instant disclosure provided lyophilized compositions (e.g., lyophilized cake) of the polypeptides disclosed herein, and methods of making the same.

Lyophilization generally includes three main stages: freezing, primary drying and secondary drying. Freezing is necessary to convert water to ice or some amorphous formulation components to the crystalline form. Primary drying is the process step when ice is removed from the frozen product by direct sublimation at low pressure and temperature. Secondary drying is the process step when bounded water is removed from the product matrix utilizing the diffusion of residual water to the evaporation surface. Product temperature during secondary drying is normally higher than during primary drying. See, Tang X. et al. (2004) “Design of freeze-drying processes for pharmaceuticals: Practical advice,” Pharm. Res., 21:191-200; Nail S. L. et al. (2002) “Fundamentals of freeze-drying,” in Development and manufacture of protein pharmaceuticals. Nail S L editors. New York: Kluwer Academic/Plenum Publishers, pp 281-353; Wang et al. (2000) “Lyophilization and development of solid protein pharmaceuticals,” M J Pharm., 203:1-60; Williams N A et al. (1984) “The lyophilization of pharmaceuticals; A literature review.” J. Parenteral Sci. Technol, 38:48-59; and WO 2010/148337 A1.

Because of the variations in temperature and pressure through the lyophilization process, an appropriate choice of excipients or other components such as stabilizers, buffering agents, bulking agents, and surfactants are needed to prevent the polypeptides disclosed herein from degradation (e.g., protein aggregation, deamidation, and/or oxidation) during freeze-drying and storage. The lyophilized compositions disclosed herein contain a particular combination of constituents allow for stable long-term storage of the polypeptides disclosed herein that comprise a circularly permuted interleukin-2 (IL-2) fused to the extracellular portion of an IL-2Rα chain.

In another aspect, the disclosure provides a lyophilized composition made by lyophilizing any one of the aqueous compositions disclosed herein that comprise a circularly permuted interleukin-2 (IL-2) fused to the extracellular portion of an IL-2Rα chain. In certain embodiments, the lyophilized composition is a lyophilized cake. In certain embodiments, the lyophilized composition is made by lyophilizing any one of the aqueous compositions disclosed herein following the lyophilization protocol recited in Table 11A or Table 11B.

In another aspect, the disclosure provides a method of making a lyophilized composition, the method comprising lyophilizing any one of the aqueous compositions disclosed herein that comprise a circularly permuted interleukin-2 (IL-2) fused to the extracellular portion of an IL-2Rα chain. In certain embodiments, the method of making a lyophilized composition comprises following the lyophilization protocol recited in Table 11A or Table 11B.

In another aspect, the disclosure provides a method of making an aqueous composition, the method comprising dissolving in an aqueous solvent any one of the lyophilized compositions disclosed herein that comprise a circularly permuted interleukin-2 (IL-2) fused to the extracellular portion of an IL-2Rα chain. In certain embodiments, the lyophilized composition is a lyophilized cake. In certain embodiments, the lyophilized composition is dissolved in 1.1 ml of water. In certain embodiments, the lyophilized composition is dissolved in 2.2 ml of water.

### Uses of Polypeptide Compositions

The compositions disclosed herein are particularly useful for the treatment, prevention, or amelioration of any disease or disorder associated with Interleukin 2 receptor signaling.

In one aspect, provided is a method of activating natural killer cells (NK) cells in a subject, the method comprising administering to the subject an effective amount of any one of the compositions disclosed herein that comprise a circularly permuted IL-2 fused to the extracellular portion of an IL-2Rα chain

In another aspect, provided is a method of treating cancer in a subject in need thereof, the method comprising administering to the subject an effective amount of any one of the compositions disclosed herein that comprise a circularly permuted IL-2 fused to the extracellular portion of an IL-2Rα chain. Cancers suitable for treatment using the composition disclosed herein include renal cell carcinoma, melanoma, ovarian cancer, and lung cancer. In certain embodiments, the cancer comprises a refractory solid tumor.

In certain embodiments, the composition is administered subcutaneously.

In certain embodiments, the composition is administered subcutaneously at a dose of about 1 mg to about 15 mg. In certain embodiments, the composition is administered subcutaneously at a dose of about 1 mg. In certain embodiments, the composition is administered subcutaneously at a dose of about 2 mg. In certain embodiments, the composition is administered subcutaneously at a dose of about 3 mg. In certain embodiments, the composition is administered subcutaneously at a dose of about 4 mg. In certain embodiments, the composition is administered subcutaneously at a dose of about 5 mg. In certain embodiments, the composition is administered subcutaneously at a dose of about 6 mg. In certain embodiments, the composition is administered subcutaneously at a dose of about 7 mg. In certain embodiments, the composition is administered subcutaneously at a dose of about 8 mg. In certain embodiments, the composition is administered subcutaneously at a dose of about 9 mg. In certain embodiments, the composition is administered subcutaneously at a dose of about 10 mg. In certain embodiments, the composition is administered subcutaneously at a dose of about 11 mg. In certain embodiments, the composition is administered subcutaneously at a dose of about 12 mg. In certain embodiments, the composition is administered subcutaneously at a dose of about 13 mg. In certain embodiments, the composition is administered subcutaneously at a dose of about 14 mg. In certain embodiments, the composition is administered subcutaneously at a dose of about 15 mg.

In certain embodiments, the composition is administered subcutaneously once a week (Q1W), once every two weeks (Q2W), or once every three weeks (Q3W).

In certain embodiments, the composition is administered subcutaneously at a dose of about 1 mg to about 15 mg once a week (Q1W), once every two weeks (Q2W), or once every three weeks (Q3W).

In certain embodiments, the composition is administered subcutaneously at a dose of about 3 mg once a week (Q1W). In certain embodiments, the composition is administered subcutaneously at a dose of about 6 mg once every three weeks (Q3W).

In certain embodiments, the melanoma is one or both of mucosal melanoma or advanced cutaneous melanoma.

It will be readily apparent to those skilled in the art that other suitable modifications and adaptations of the methods described herein may be made using suitable equivalents without departing from the scope of the embodiments disclosed herein.

Having now described certain embodiments in detail, the same will be more clearly understood by reference to the following examples, which are included for purposes of illustration only and are not intended to be limiting.

### EXAMPLES

The invention is further illustrated by the following examples, which should not be construed as further limiting. The practice of the present invention will employ, unless otherwise indicated, conventional techniques of organic synthesis, cell biology, cell culture, molecular biology, transgenic biology, microbiology and immunology, which are within the skill of the art.

### Example 1—Design and Testing of Polypeptide Compositions

In an effort to determine the optimal subcutaneous formulation for Polypeptide A (a circularly permuted IL-2 fused to the extracellular portion of an IL-2Rα chain comprising the amino acid of SEQ ID NO: 1), several formulations of Polypeptide A were tested for their effects on protein stability, pH stability, physio-chemical behavior, lyophilized cake uniformity, and resistance to adhering to the storage vial post-lyophilization. Two main objectives were sought in this study. The first objective was to produce a lyophilized cake which, when reconstituted with off-the shelf diluent (i.e. water for injection), results in an isotonic solution ready for administration. A formulation that results in a non-isotonic solution when reconstituted with WFI would not be amenable to subcutaneous administration. The second objective was to produce a lyophilized cake with optimal cake appearance that has minimal cake shrinkage. An improved cake appearance may make the drug product more visually appealing to a patient or healthcare provider, thus potentially improving compliance in drug use. Table 1 below recites the specific components and their concentrations for the Polypeptide A formulation that was initially designed for intravenous administration.

TABLE 1
Polypeptide A intravenous administration formulation

| Component | Function | Concentration (mg/mL) |
|---|---|---|
| Polypeptide A | Protein | 1, 5 and 15 |
| Sucrose | Protein Stabilizer | 50 |
| Sodium citrate dihydrate | Buffer | 2.03 |
| Citric acid monohydrate | Buffer | 0.97 |
| Polysorbate 20 | Surfactant | 0.1 |

| | |
|---|---|
| Buffer pH | 6.1 |
| Osmolarity in WFI (mOsm/kg) | ~125 mOsm/kg |

### Analytical Methods Used

### Fourier Transform Infrared (FTIR) Spectroscopy

FTIR analysis was performed using PROTA FTIR Protein Analyzer equipped with a CaF2 Biocell and ATR cell for solid sample analysis. Approximately 10 μL of liquid sample or 10 mg of lyophilized powder sample was loaded for the analysis. Absorbance signals were processed by subtracting interfering signals from background lyophilized placebo and buffer where appropriate. The processed data were finally converted to second derivative signals to improve the resolution with parameters set as 100 scans with a 4 cm⁻¹ resolution. To identify the percentage of the different structural elements of the Polypeptide A native protein, the subtracted spectrum was processed through the protein secondary structure database. The percent similarity between the native state and the dried state was calculated based on the area of overlap from 1700 to 1600 cm⁻¹ or 1800 to 1400 cm⁻¹.

### Osmolality Analysis

The osmolality of prepared formulations were determined using wescor vapro vapor pressure instrument. For each analysis, approximately 10 μL of liquid sample was utilized.

### Visual Inspection

All vials were inspected against a background that provided a clear picture when digital photos were captured.

### Sub-Ambient and Dry Powder Differential Scanning Calorimetry (DSC)

DSC analysis was performed using TA Q20 with a refrigerated cooling system I. For sub-ambient (frozen state) DSC, approximately 15 μL of the formulated drug substance was loaded into DSC pan and hermetically sealed. The sample was then cooled to −90° C. at 10° C./minute. The pans were held in the sample chamber for 2 minutes prior to warming to 30° C. at 10° C./minute. During annealing, the sample was warmed to −10° C. after holding at −90° C. for 2 minutes, then cooled back to −90° C. prior to warming to 30° C. at 10° C./minute.

For dry powder high temperature DSC, vials were placed into a dry box that was purged to below 8% RH with dry air. Aliquots of the lyophilized samples were removed and hermetically sealed in DSC pans. Thermal scanning was carried out from 20° C. to 180° C. at 1° C./minute using a modulation program of ±1° C. every 120 seconds and the resulting reversing and non-reversing heat flow measured.

### Screening Study 1

The first screening involved various formulations to understand how different combinations of stabilizers, bulking agents, etc. would impact the glass transition or collapse temperature (Tg′) and the isotonicity. All formulations contained 12 mM sodium citrate buffer at pH 6.11 as the base formulation prior to the addition of the screening excipients. Tween 21 was used as a surface stabilizer. The formulations evaluated in this study with sub-ambient DSC and osmolality analyses are summarized in Table 2.

TABLE 2
Screening Study 1 formulations

| Formulation | Polypeptide | Sucrose | Mannitol | Osmolality | Tg′ |
|---|---|---|---|---|---|
| # | A (mg/mL) | (mg/mL) | (mg/mL) | (mOsm/kg) | ° C. |
| 1 | 5 | 5 | 15 | 123 | −28.81 |
| 2 | 5 | 5 | 10 | Not | −27.91 |
| | | | | measured | |
| 3 | 5 | 10 | 10 | 66 | −28.00 |
| 4 | 5 | 5 | — | 59 | −25.70 |

The osmolality values for Formulations 1-4 were too low, a value closer to physiological (280-320 mOsm/kg) was desired. Formulation 1 showed signs of being metastable in the frozen phase; hence the lyophilization cycle was annealed at −10° C., which successfully converted this metastable state to a stable eutectic phase. In an attempt to increase the osmolality of these formulations, glycine was added to Formulation 4 at a concentration of 30 mg/mL. This resulted in an osmolality of 413 mOsm/kg. Based on this result, glycine formulations were prepared to replace Formulations 1 and 2 to target osmolality of 290 mOsm/kg, with a corresponding glass transition temperature of −28.23° C. by DSC. Additionally, concentrations of sucrose and mannitol excipients in Formulations 3 and 4 were adjusted to increase the osmolality values. Four new formulations were lyophilized and analyzed in screening study 2.

### Screening Study 2

The formulations tested in screening study 2 and their associated osmolality values are shown below in Table 3. All formulations contained 12 mM sodium citrate buffer at pH 6.11 as the base formulation prior to the addition of the screening excipients. Tween 21 was used as a surface stabilizer. Note that placebos were prepared for each formulation to be used for secondary structure analysis following lyophilization in a VirTis Genesis SQ Super XL-70 freeze dryer.

TABLE 3
Screening Study 2 formulations with Osmolality and Percent Similarity of
Secondary Structure by FTIR

| Formulation | Polypeptide | Sucrose | Mannitol | Glycine | Osmolality | % similarity |
|---|---|---|---|---|---|---|
| # | A (mg/mL) | (mg/mL) | (mg/mL) | (mg/mL) | (mOsm/kg) | to native |
| 5 | 5 | 5 | — | 15 | 268 | 52.6 |
| 6 | 5 | 10 | — | 15 | 248 | 65.3 |
| 7 | 5 | 5 | 30 | — | 206 | 51.9 |
| 8 | 5 | 10 | 30 | — | 224 | 80.6 |

All samples were sterile filtered under aseptic conditions followed by filling into sterilized 2 cc vials at fill volumes of 0.5 mL. Table 4 details lyophilization cycle parameters used for screening study 2.

TABLE 4
Lyophilization Cycle Parameters for Screening Study 2

| | Temperature | Time | Ramp Rate | Chamber Pressure |
|---|---|---|---|---|
| Step | (° C.) | (Minutes) | (° C./min) | (mTorr) |
| Loading | 5 | — | — | — |
| Freezing and | 5 to −40 | 100 | 0.5 | — |
| Annealing | Hold at −40 | 30 | — | — |
| | −40 to −10 | 60 | 0.5 | — |
| | Hold at −10 | 60 | — | — |
| | −10 to −40 | 60 | 0.5 | — |
| | Hold at −40 | 60 | — | 100 |
| Primary | −40 to −25 | 30 | 0.5 | 100 |
| Drying | Hold at −25 | 600 | — | 100 |
| Secondary | −25 to 10 | 100 | 0.5 | 100 |
| Drying | Hold at 10 | 60 | — | 100 |

| | | |
|---|---|---|
| Stoppering | 10 | Sealed under partial vacuum |
| | | (nitrogen back fill) |

Following lyophilization, all lyophilized cakes showed no sign of melt-back or collapse (no images were taken). The samples were tested for secondary structure and compared to the native state structure collected in the liquid cell. FTIR spectra (second derivative) of native Polypeptide A were overlaid with screening study 2 formulations. The percent similarity between the native and the dried state shown in Table 3 was calculated based on the second derivative area of overlap from 1800 to 1400 cm-1. Formulation 8 showed the best retention of secondary structure after drying, however the tonicity was low.

### Screening Study 3

Based on the above results, a third screening study was conducted to investigate different ratios of stabilizer vs. bulking agent. In addition, trehalose, a non-reducing disaccharide and polyvinyl pyrrolidone (PVP), a polymer, were introduced to evaluate their effect as stabilizers and also to evaluate its effectiveness to improve Tg′. Eight formulations were prepared for screening study 3 and freeze-dried using lyophilization cycle parameters described in Table 4. The formulation matrix for this study is detailed in Table 5 below. Tween 20 (PS20) was used in this study at 0.1 mg/mL for Formulations 9-16.

TABLE 5
Screening Study 3 formulations with Percent Similarity of Secondary Structure
by FTIR

| Formulation | Polypeptide | Sucrose | Trehalose | Mannitol | PVP | % similarity |
|---|---|---|---|---|---|---|
| # | A (mg/mL) | (mg/mL) | (mg/mL) | (mg/mL) | (mg/mL) | to native |
| 9 | 5 | 5 | — | 30 | — | 40.3 |
| 10 | 5 | 10 | — | 30 | — | 73.7 |
| 11 | 5 | 15 | — | 20 | — | 61.5 |
| 12 | 5 | 15 | — | 20 | 10 | 45.4 |
| 13 | 5 | 20 | — | 20 | — | 58.4 |
| 14 | 5 | — | 15 | 15 | — | 81.2 |
| 15 | 5 | — | 15 | 15 | 10 | 76.0 |
| 16 | 5 | — | 20 | 15 | — | 71.8 |

FTIR spectra were measured as described above to determine the percent similarity to native Polypeptide A. In addition to Formulation 8, the formulations showing the most promising results with >70% similarity were Formulations 10, 14, 15, and 16. Based on this observation, screening study 4 was designed with five formulations prepared with lower amounts of mannitol, and higher amounts of disaccharide. This combination appeared to favor a higher retention of secondary structure.

### Screening Study 4

Table 6 depicts 5 formulations that were prepared as described above for screening study 3 and freeze-dried using lyophilization cycle parameters described in Table 4. No osmolality measurements were performed. Tween 20 (PS20) was used in this study at 0.1 mg/mL for Formulations 17-21.

TABLE 6
Screening Study 4 formulations with Percent Similarity of
Secondary Structure by FTIR

| | | | | | % |
|---|---|---|---|---|---|
| Formulation | Polypeptide | Sucrose | Trehalose | Mannitol | similarity |
| # | A (mg/mL) | (mg/mL) | (mg/mL) | (mg/mL) | to native |
| 17 | 5 | 15 | — | 10 | 78.4 |
| 18 | 5 | 15 | — | 5 | 84.5 |
| 19 | 5 | — | 15 | 10 | 64.5 |
| 20 | 5 | — | 15 | 5 | 67.5 |
| 21 | 5 | — | 25 | — | 95.2 |

FTIR spectra were measured as described above to determine the percent similarity to native Polypeptide A. Based on the above results, a higher amount of disaccharide was confirmed to have a higher impact on the retention of secondary structure. The next screening study was designed to investigate higher concentrations of disaccharides in combination with bulking agents.

### Fine Tuning the Tonicity

Prior to the start of the next screening, the isotonicity of various formulations were fine tuned. A number of formulations containing excipient combinations were prepared in 12 mM sodium citrate buffer at pH 6.1 with higher amounts of disaccharides and varying amounts of bulking agents, followed by osmolality measurement. The different formulations and the resulting osmolality values are shown in Table 7. The formulations all contain Polypeptide A at 5 mg/mL.

TABLE 7
Osmolality screening

| Formulation | Sucrose | Trehalose | Mannitol | Glycine | Osmolality |
|---|---|---|---|---|---|
| # | (mg/mL) | (mg/mL) | (mg/mL) | (mg/mL) | (mOsm/kg) |
| 22 | 20 | — | — | 40 | 612 |
| 23 | 25 | — | — | 15 | 303 |
| 24 | 30 | — | — | 10 | 258 |
| 25 | — | 25 | 10 | — | 164 |
| 26 | — | 25 | 20 | — | 209 |
| 27 | — | 30 | 30 | — | 289 |
| 28 | — | 30 | — | 20 | 372 |
| 29 | — | 20 | — | 20 | 293 |
| 30 | — | 25 | — | 15 | 303 |

### Screening Study 5

Table 8 depicts 5 formulations that were prepared as described above for screening study 3. Tween 20 (PS20) was used in this study at 0.1 mg/mL for Formulations 31-35. Glycine in Formulations 33 and 34 interfered with the FTIR analysis, hence there is no percent similarity data reported. Moreover, generating an effective lyophilization cycle with glycine was more difficult than mannitol. Therefore, mannitol-containing formulations were pursued further.

TABLE 8
Screening Study 5 formulations with Osmolality and Percent Similarity of
Secondary Structure by FTIR

| Formulation | Sucrose | Trehalose | Mannitol | Glycine | Osmolality | % similarity |
|---|---|---|---|---|---|---|
| # | (mg/mL) | (mg/mL) | (mg/mL) | (mg/mL) | (mOsm/kg) | to native |
| 31 | — | 30 | 30 | — | 299 | 80.8 |
| 32 | 30 | — | 30 | — | 300 | 77.9 |
| 33 | — | 30 | — | 10 | 252 | — |
| 34 | — | 20 | — | 15 | 292 | — |
| 35 | — | 20 | 35 | — | 282 | 89.0 |

All samples were sterile filtered under aseptic conditions followed by filling into sterilized 2 cc vials at fill volumes of 0.5 mL. Table 9 details lyophilization cycle parameters used for screening study 5.

TABLE 9
Lyophilization Cycle Parameters for Screening Study 5

| | Temperature | Time | Ramp Rate | Chamber Pressure |
|---|---|---|---|---|
| Step | (° C.) | (Minutes) | (° C./min) | (mTorr) |
| Loading | 5 | — | — | — |
| Freezing and | 5 to −45 | 100 | 0.5 | — |
| Annealing | Hold at −45 | 30 | — | — |
| | −45 to −10 | 70 | 0.5 | — |
| | Hold at −10 | 40 | — | — |
| | −10 to −45 | 70 | 0.5 | — |
| | Hold at −45 | 30 | — | 90 |
| Primary | −45 to −30 | 30 | 0.5 | 90 |
| Drying | Hold at −30 | 1260 | — | 90 |
| Secondary | −30 to 10 | 100 | 0.5 | 90 |
| Drying | Hold at 10 | 60 | — | 90 |

| | | |
|---|---|---|
| Stoppering | 10 | Sealed under partial vacuum |
| | | (nitrogen back fill) |

After freeze-drying, the secondary structure was determined and compared to the native state as described above and their percent similarity calculated.

### Leading Formulation Candidates

Based in part on FTIR and osmolality results, Formulations 31 and 32 were tested for appearance, glass transition temperature in the frozen state (Tg′), and glass transition temperature in the dried state (Tg). The intravenous administration formulation for Polypeptide A was used as a comparator. Compositions of the three formulations evaluated in this screen are described in Table 10.

TABLE 10
Formulations with Sub-Ambient Glass Transition Temperature

| Formulation | Polypeptide | Polysorbate | Sucrose | Trehalose | Mannitol | Tg′ |
|---|---|---|---|---|---|---|
| # | A (mg/mL) | 20 (mg/mL) | (mg/mL) | (mg/mL) | (mg/mL) | (° C.) |
| 36 | 5 | 0.1 | 50 | — | — | −29.42 |
| 37 (31 in | 5 | 0.1 | 30 | — | 30 | −38.39 |
| Table 8) | | | | | | |
| 38 (32 in | 5 | 0.1 | — | 30 | 30 | −37.83 |
| Table 8) | | | | | | |

All formulations (placebo and active) were prepared and sterile filtered under aseptic conditions followed by filling into sterilized 5 cc vials at fill volumes of 2.28 mL. An aliquot (15 μL) of each formulation candidate was analyzed by sub-ambient DSC to assess their frozen state profiles. Based on the Tg′ results, the formulations were lyophilized using cycle parameters listed in Table 11A. An annealing step is used to ensure complete crystallization of the bulking agent, mannitol. An alternative lyophilization cycle which was similarly effective at lyophilizing the formulations is recited below in Table 11B.

TABLE 11A
Lyophilization Cycle Parameters for Leading Formulation Candidates

| | Temperature | Time | Ramp Rate | Chamber Pressure |
|---|---|---|---|---|
| Step | (° C.) | (Minutes) | (° C./min) | (mTorr) |
| Loading | 5 | — | — | — |
| Freezing and | 5 to −42 | 94 | 0.5 | — |
| Annealing | Hold at −42 | 30 | — | — |
| | −42 to −10 | 64 | 0.5 | — |
| | Hold at −10 | 60 | — | — |
| | −10 to −40 | 20 | 0.5 | — |
| | Hold at −40 | 60 | — | 100 |
| Primary | −45 to −30 | 20 | 0.5 | 100 |
| Drying | Hold at −30 | 2580 | — | 100 |
| Secondary | −30 to 25 | 110 | 0.5 | 100 |
| Drying | Hold at 25 | 540 | — | 100 |

| | | |
|---|---|---|
| Stoppering | 25 | Sealed under partial vacuum |
| | | (nitrogen back fill) |

TABLE 11B
Alternative Lyophilization Cycle Parameters

| | Temperature | Time | Chamber Pressure |
|---|---|---|---|
| Step | (° C.) | (Minutes) | (Torr) |
| Loading | 5 | — | 760 |
| Loading | 5 | 30 | 550 |
| Freezing and | 5 to −45 | 150 | 550 |
| Annealing | Hold at −45 | 120 | 550 |
| | −45 to −10 | 105 | 550 |
| | Hold at −10 | 180 | 550 |
| | −10 to −45 | 105 | 550 |
| | Hold at −45 | 120 | 550 |
| Primary | Hold at −45 | 30 | 0.1 |
| Drying | −45 to −25 | 40 | 0.1 |
| | Hold at −25 | 3000 | 0.1 |
| Secondary | −25 to 30 | 220 | 0.1 |
| Drying | Hold at 30 | 1440 | 0.1 |
| Stoppering | 30 | — | 600 |

Following lyophilization, all samples were evaluated based on cake appearance, glass transition temperature in the dried state (Tg), pH, reconstitution time, osmolality, concentration, moisture content, SEC, RP and potency assay.

### Lyophilized Cake Appearance

The vials contained white intact cakes. The formulation containing 50 mg/mL sucrose (Formulation 36) displayed slight cake shrinkage. All other formulations were fully intact with no signs of shrinkage.

### High Temperature DSC of Lyophilized Cake

Table 12 summarizes the results after high temperature DSC analysis. Glass transition temperature (Tg) values for Formulation 36 and 37 were determined at −82° C., attributed to sucrose. The glass transition of the formulation containing trehalose (Formulation 38) could not be detected under these conditions. The melting peak detected for Formulation 36 at 158° C. was attributed to the melting of sucrose. The melting peaks detected for Formulation 37 and 38 at 120-121° C. were attributed to the melting of mannitol.

TABLE 12
Summary of Tg and Melting Temperature

| | | Glass Transition | Melting |
|---|---|---|---|
| | | Temperature, | Temperature, |
| | Sample weight | Tg | Tm |
| Formulation # | (mg) | (° C.) | (° C.) |
| 36 | 4.5 | 81.86 | 157.86 |
| 37 (31 in Table 8) | 5.2 | 81.86 | 119.69 |
| 38 (32 in Table 8) | 4.9 | — | 120.80 |

### pH, Reconstitution Time, Osmolality, Concentration, and Moisture Content for Leading Formulation Candidates

WFI was used to reconstitute the lyophilized cake. Table 13 summarizes the results for pH, Reconstitution Time, Osmolality, Concentration, and Moisture Content.

TABLE 13
Summary of Tg and Melting Temperature

| | | | Recon. Time | | |
|---|---|---|---|---|---|
| | | | with stirring | Moisture | |
| Formulation | | Osmolality | and swirling | Content | Concentration |
| # | pH | (mOsm/kg) | (seconds) | (%) | (mg/mL) |
| 36 | 6.18 | 188 | 20 | 1.55 | 5.127 |
| 37 (31 in | 6.20 | 296 | 22 | 1.74 | 5.058 |
| Table 8) | | | | | |
| 38 (32 in | 6.17 | 289 | 16 | 1.79 | 4.566 |
| Table 8) | | | | | |

### Size Exclusion (SE) HPLC and Reverse Phase (RP) HPLC for Leading Formulation Candidates

SE-HPLC and RP-HPLC analysis of Formulations 36, 37, and 38 was performed. The integrated results for the SE-HPLC analysis showed the largest peak having a peak area of >98% for each formulation. The integrated results for the RP-HPLC analysis showed the largest peak having a peak area of >85% for each formulation.

### Potency Assay for Leading Formulation Candidates

To ensure that the excipients would not impact bioactivity, Formulations 36-38 were compared using a pSTATS Activity Assay. The dose response curves were similar and comparable to that of a Polypeptide A Reference Standard. Data analysis in Table 14 showed EC50 values that were greater than 75% potency relative to the Reference Standard RT, which is acceptable.

TABLE 14
pSTAT5 activity assay results

| Parameter | Formulation 36 | Formulation 37 | Formulation 38 |
|---|---|---|---|
| Bottom Asymptote | 0.116 | 0.109 | 0.072 |
| Top Asymptote | 0.757 | 0.786 | 0.755 |
| Hill Slope | 1.29 | 1.24 | 1.34 |
| EC50 | 11.59 | 12.49 | 16.07 |
| Relative Potency | 84% | 78% | 78% |

The pSTATS activity assay was performed by measuring binding of the formulations to HH cells (a human T lymphocyte cell line which have the βγIL2 receptor isoform present on their surface). Polypeptide A binding was measured by determining the amount of phosphorylated STATS (phospho-STATS or pSTATS) present in the HH cells after contact with each formulation, using an ELISA assay. The Invitrogen InstantOne ELISA phosphor-STATS alpha/beta (pTyr694/pTyr699) kit was used to perform the ELISA assay.

The drug product sample was prepared by reconstituting the sample in 2.2 mL WFI. The sample was visually inspected to ensure that contents were free of visible particulates.

A sample diluent was prepared by adding 25 mL of Fetal Bovine Serum (FBS) to 500 mL of Hanks Balanced Salt Solution (HBSS) for a final concentration of 5% FBS and warmed to 37° C. A wash buffer comprising Phosphate Buffered Saline (PBS) with 0.05% Tween 20 was used.

Samples and standards were diluted to final protein concentrations in the assay of 750 ng/mL, 250 ng/mL, 83 ng/mL, 28 ng/mL, 9.3 ng/mL, 3.1 ng/mL, 1.0 ng/mL, and 0.3 ng/mL. A stock solution of HH cells at a density of approximately 1.2×10⁶ cells/mL was prepared and 50 μl of the cell stock solution was added to each well of a 96-well plate that contained the diluted sample or standard. The cells were incubated at 37° C. for 30 minutes. After incubation, the cells were lysed in a cell lysis buffer for 10 minutes. After lysis, 50 μl of the lysed cell mix was transferred to an ELISA plate followed by 50 μl of phospho-STATS A/B antibody cocktail. The mix was then incubated for 1 hour then washed 3 times with wash buffer. 100 μl of a detection reagent was then added to each well and the plate was incubated for 15 minutes. 100 μl of a stop solution was then added to each well and the plate was read at 450 nm on a microplate reader.

Individual EC50 values were measured and % relative standard deviation (RSD) was calculated for the reference standard EC50 values and for the control EC50 values.

The geometric mean of the three EC50 values for reference standard (Ref Std EC50GM) and the geometric mean of three EC50 values for the control (Control EC50) were calculated. The relative potency for the control was calculated using the following equation: Relative Potency=(RefStd EC50GM)/(Control EC50)×100%.

The samples were calculated in the same manner. Results of the assay were determined by the following equation: Relative Potency=(RefStd EC50GM)/(Test sample EC50)×100%.

### INITIAL CONCLUSIONS

Based on all the results generated and its similarity to the intravenous administration Polypeptide A formulation, which also contains sucrose as the stabilizing agent, Formulation 37 was selected. The formulation composition is shown in Table 15. The selected formulation results in an isotonic solution when reconstituted using WFI, which will increase its usability for direct subcutaneous drug delivery.

The screening studies suggested that the preservation of Polypeptide A secondary structure is improved significantly when high amounts of disaccharides such as sucrose or trehalose is present in the formulation. Additionally, mannitol was determined to be a superior bulking agent and tonicity modifier compared to glycine.

TABLE 15
Polypeptide A Formulation 37

| Component | Function | Concentration (mg/mL) |
|---|---|---|
| Polypeptide A | Protein | 1 and 5 |
| Sucrose | Protein Stabilizer | 30 |
| Mannitol | Bulking Agent | 30 |
| Sodium citrate tribasic | Buffer | 2.03 |
| dihydrate | | |
| Citric acid monohydrate | Buffer | 0.97 |
| Polysorbate 20 | Surfactant | 0.1 |

Following identification of Formulation 37 recited above, the concentrations of sodium citrate tribasic dihydrate and citric acid monohydrate were optimized to avoid a pH titration step to achieve a pH of 6.1. Table 16 below describes altered Formulation 37-2.

TABLE 16
Polypeptide A Formulation 37-2

| Component | Function | Concentration (mg/mL) |
|---|---|---|
| Polypeptide A | Protein | 1 and 5 |
| Sucrose | Protein Stabilizer | 30 |
| Mannitol | Bulking Agent | 30 |
| Sodium citrate tribasic | Buffer | 2.91 |
| dihydrate | | |
| Citric acid monohydrate | Buffer | 0.34 |
| Polysorbate 20 | Surfactant | 0.1 |

### Further Testing of Mannitol/Sucrose Levels

The amounts of sucrose and mannitol were altered from Formulation 37 as shown in Table 17 to test osmolality and monitor lyophilized cake appearance. Each formulation below in Table 17 had a sodium citrate buffer at 12 mM and a pH of 6.1. Following lyophilization, Formulation 37 had the best lyophilized cake appearance, showing more of a concave top appearance and no shrinkage on the walls compared to the other formulations. The improved cake appearance may make the drug product more visually appealing to a patient or healthcare provider, thus potentially improving compliance in drug use.

TABLE 17
Formulations with Altered Levels of Mannitol and Sucrose

| Sample | Formulation | Polypeptide | Polysorbate | Sucrose | Mannitol | Osmolality |
|---|---|---|---|---|---|---|
| # | # | A (mg/mL) | 20 (mg/mL) | (mg/mL) | (mg/mL) | (mOsm/kg) |
| 1 | 37 | 5 | 0.1 | 30 | 30 | 285 |
| 2 | 39 | 5 | 0.1 | 20 | 40 | 311 |
| 3 | 40 | 5 | 0.1 | 13.5 | 40 | 292 |
| 4 | 41 | 5 | 0.1 | 10 | 40 | 281 |
| 5 | 42 | 5 | 0.1 | 90 | — | 296 |
| 6 | 36 | 5 | 0.1 | 50 | — | 179 |

1. A composition comprising:
a) about 1 mg to about 50 mg of a polypeptide comprising a circularly permuted IL-2 fused to the extracellular portion of an IL-2Rα chain;
b) sucrose;
c) mannitol;
c) citrate buffer; and
d) an emulsifier.

2. The composition of claim 1, wherein:
the polypeptide comprises an amino acid sequence having at least 95% identity to SEQ ID NO: 1;
the composition comprises about 1 mg to about 15 mg of the polypeptide, optionally about 1 mg, about 5 mg, about 15 mg, about 20 mg, or about 30 mg of the polypeptide;
the composition comprises about 60 mg to about 72 mg sucrose, optionally about 66 mg sucrose;
the composition comprises about 60 mg to about 72 mg mannitol, optionally about 66 mg mannitol;
the composition comprises about 4.0 mg to about 6.0 mg citrate anion, optionally about 5.0 mg citrate anion;
the composition comprises citric acid and sodium citrate tribasic dihydrate in a mass ratio of citric acid:sodium citrate tribasic dihydrate of between about 1:10 to about 1:2, optionally about 1:9 or about 1:2; and/or
the emulsifier comprises polysorbate 20, optionally about 0.10 mg to about 0.12 mg polysorbate 20, optionally about 0.11 mg polysorbate 20.

3-21. (canceled)

22. The composition of claim 1, wherein the composition is a lyophilized cake, optionally wherein:
dissolution of the lyophilized cake in water results in an aqueous solution with a pH of about 5.5 to about 6.5, optionally with a pH of about 6.1;
wherein dissolution of the lyophilized cake in water results in an aqueous solution with an isotonic osmolality; and/or
wherein dissolution of the lyophilized cake in water results in an aqueous solution with an osmolality of about 240 to about 340 mOsm/kg, optionally about 285 mOsm/kg or about 300 mOsm/kg.

23-29. (canceled)

30. The composition of claim 1, wherein the composition is an aqueous solution, optionally wherein:
the composition comprises about 0.5 mg/mL to about 30 mg/mL of the polypeptide, optionally wherein:
the composition about 1 mg/mL of the polypeptide, optionally the composition is a 1.1 ml aqueous solution comprising about 1.1 mg of the polypeptide;
the composition comprises about 5 mg/mL of the polypeptide, optionally the composition is a 1.1 ml aqueous solution comprising about 15 mg of the polypeptide;
the composition comprises about 20 mg/mL of the polypeptide; or
the composition comprises about 30 mg/mL of the polypeptide.

31-37. (canceled)

38. The composition of claim 30, wherein:
the composition comprises about 25 mg/mL to about 35 mg/mL sucrose, optionally about 30 mg/mL sucrose;
the composition comprises about 25 mg/mL to about 35 mg/mL mannitol, optionally about 30 mg/mL mannitol;
the composition comprises about 10 mM to about 20 mM citrate buffer, optionally about 12 mM citrate buffer;
the composition comprises about 0.09 mg/mL to about 0.11 mg/mL polysorbate 20, optionally about 0.1 mg/mL polysorbate 20; and/or
the pH of the composition is about 5.5 to about 6.5, optionally about 6.1.

39-43. (canceled)

44. The composition of claim 30, wherein:
the citrate buffer is formed by the combination of 2.03 mg/mL sodium citrate tribasic dihydrate and 0.97 mg/mL citric acid monohydrate in the aqueous solution;
the citrate buffer is formed by the combination of 2.91 mg/mL sodium citrate tribasic dihydrate and 0.34 mg/mL citric acid monohydrate in the aqueous solution;
the citrate buffer is formed by the combination of 2.96 mg/mL sodium citrate tribasic dihydrate and 0.30 mg/mL citric acid monohydrate in the aqueous solution.

45-50. (canceled)

51. The composition of claim 30, wherein the osmolality of the composition is about 240 to about 340 mOsm/kg, about 280 to about 320 mOsm/kg, about 285 mOsm/kg, or about 300 mOsm/kg.

52-54. (canceled)

55. The composition of claim 30, wherein the aqueous solution comprises about 0.03 mg/mL of the polypeptide to about 0.2 mg/mL of the polypeptide.

56. A lyophilized composition made by lyophilizing the composition of claim 30.

57. The composition of claim 1, wherein the composition is a single unit dose of the polypeptide.

58. An article of manufacture comprising the composition of claim 1, optionally wherein the article is a glass vial.

59. (canceled)

60. A method of making a lyophilized composition, the method comprising lyophilizing the aqueous solution of claim 30.

61. A method of making an aqueous composition, the method comprising dissolving the composition of claim 22 in an aqueous solvent, optionally wherein:
the pH of the aqueous composition is adjusted to about 6.1, optionally adjusted to about 6.1 with a base, such as sodium hydroxide;
the aqueous composition is further diluted with an aqueous solution comprising about 1% (w/w) of a surfactant, optionally wherein the surfactant is polysorbate 20; and/or
the aqueous solution further comprises about 0.1% (w/w) citric acid monohydrate, 0.2% (w/w) sodium citrate tribasic dihydrate, and 98.7% (w/w) water for injection.

62-67. (canceled)

68. A method of activating natural killer cells (NK) cells in a subject, the method comprising administering to the subject an effective amount of the composition of claim 30.

69. A method of treating cancer in a subject in need thereof, the method comprising administering to the subject an effective amount of the composition of claim 30, optionally wherein:
the cancer is renal cell carcinoma, melanoma, ovarian cancer, or lung cancer, optionally wherein the melanoma is one or both of mucosal melanoma or advanced cutaneous melanoma; and/or
the cancer comprises a refractory solid tumor.

70-71. (canceled)

72. The method of claim 69, wherein the composition is administered subcutaneously, optionally wherein:
the composition is administered subcutaneously at a dose of about 1 mg to about 15 mg; or
the composition is administered subcutaneously at a dose of about 1 mg to about 15 mg once a week (Q1W), once every two weeks (Q2W), or once every three weeks (Q3W).

73-75. (canceled)

76. A method of treating melanoma in a subject in need thereof, the method comprising administering to the subject an effective amount of a composition comprising:
a) about 1 mg to about 50 mg of a polypeptide comprising a circularly permuted IL-2 fused to the extracellular portion of an IL-2Rα chain;
b) sucrose;
c) mannitol;
c) citrate buffer; and
d) an emulsifier.

77. The method of claim 76, wherein the polypeptide comprises an amino acid sequence having at least 95% identity to SEQ ID NO: 1.

78. The method of claim 76, wherein the melanoma is one or both of mucosal melanoma or advanced cutaneous melanoma.

79. The method of claim 76, wherein the composition is administered subcutaneously at a dose of about 1 mg to about 15 mg once a week (Q1W), once every two weeks (Q2W), or once every three weeks (Q3W).
